(19) United States
(12) Patent Application Publication
(10) Pub. No.: US 2004/0018532 A1
(43) Pub. Date: Jan. 29, 2004

(54) Electronic sensor device

(75) Inventor: Filip Frederix, Hasselt (BE)

Correspondence Address: KNOBBE MARTENS OLSON & BEAR LLP, 2040 MAIN STREET, FOURTEENTH FLOOR, IRVINE, CA, 92614, US

(73) Assignee: Interuniversitair Microelektronica Centrum (IMEC), Leuven (BE)

(21) Appl. No.: 10/454,765

(22) Filed: Jun. 3, 2003

Related U.S. Application Data
(60) Provisional application No. 60/412,100, filed on Sep. 19, 2002.
(60) Provisional application No. 60/385,869, filed on Jun. 4, 2002.

(30) Foreign Application Priority Data
Oct. 25, 2002 (EP) 02447203.7

(51) Int. Cl. C12Q 1/68; C12M 1/34

(52) U.S. Cl. 435/6; 435/287.2

(57) ABSTRACT

The present invention is related to a device suitable for the preparation of a sensor, comprising a substrate comprising a metal layer, the metal layer comprising at least a first region wherein to a first region is attached a first species comprising a compound of chemical formula:

X—R₁—S—S—R₂—Y

wherein R₁ and R₂ represent independently from each other spacer of n carbon atoms, n being an integer higher than 11; wherein X represents:

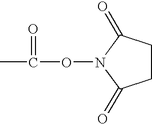

and Y represents an organic group. The first species is able to form a self-assembling monolayer on the first region.

### RELATED APPLICATION

This application claims the benefit of U.S. Provisional Application No. 60/385,869, filed Jun. 4, 2002, and U.S. Provisional Application No. 60/412,100, filed Sep. 19, 2002.

### FIELD OF THE INVENTION

The present invention is related to an electronic sensor device and in particular to devices including self-assembled monolayers bounded to a recognition molecule to perform highly sensitive and selective analysis. Furthermore the present invention is related to a method for the preparation of such monolayers and corresponding sensors.

### BACKGROUND OF THE INVENTION

Health and environment related fields, faces various biochemical processes which have to be evaluated rapidly at decreasing detection levels. Many biochemical analytical methods involve immobilization of a biological molecule on a surface.

The increasing miniaturization and the demand for sensitivity require a covalent immobilization of biomolecules. Affinity biosensor transducers are defined as systems containing at least one biological element able to recognize an analyte. This element is called the biological recognition layer.

The biological recognition layer consists of a probe molecule, covalently bound to a linking layer, which makes the connection with the transducer. The concentration of this analyte is translated by an electrical signal via the right combination of an efficient biological recognizer and an adequate translation system.

Such sensors combine the extremely high biochemical selectivity with the speed of micro-electronic transducers.

The presence of analytes can be detected by sensing fluids using acoustic waves as described in U.S. Pat. No. 4,361,026. The solid phase refers to any material insoluble in a medium containing a target molecule. The substrate can be a deposit of a metal film on any convenient support or any other solid surface able to selectively bind monolayers. Preferred metals include gold, silver, GaAs, palladium, platinum, copper, and the like.

Silanes and alkyl phosphate monolayers can also be used on oxide material supports like SiO₂, Nb₂O₅, TiO₂, ZrO₂, Al₂O₃, and Ta₂O₅.

A biosensor must respond to major qualities like stability, specificity, selectivity, and reproducibility.

For all those reasons, affinity biosensors are not yet commercially available. The major challenge being the realization of new specific and selective self-assembled monolayers and the receptors. An analyte must be detectable in an excess of other proteins.

The most common receptors are antibodies and specific binding proteins which have a reversible specific binding affinity for an analyte. Chemical modifications of the surface moieties may create new surface functionalities, such as, for example, amine-terminated functional groups appropriate for particular diagnostic or therapeutic operations.

Bamdad et al. in U.S. Pat. Nos. 5,620,850 and 6,127,129 discloses a biosensor of a formula X—R-Ch-M adhered to a surface as part of a self assembled monolayer, where X is a functionality that adheres to the surface, R is a spacer moiety and Ch is a chelating agent for the metal ion M. The monolayers described in this patent only have limited surface accessibility for biological binding and oriented immobilization. Moreover, the monolayers can only be achieved via an extra crosslinker step.

Lahiri et al. in Anal. Chem. 1999, 71, 777-790 describe a method for immobilizing proteins on mixed self-assembling monolayers of alkanethiols. The method includes the steps of obtaining a N-hydroxysuccinimidyl (NHS) ester from the carboxylic acid groups of the self-assembling monolayer and coupling this ester to a free amine group of the protein. In a first step, a self-assembling monolayer is formed on a gold surface. The self-assembling monolayer has free carboxylic acid groups. In a next step, the surface carboxylic acid groups are activated with NHS and ethylene dichloride (EDC) to form the NHS ester and displacement of the NHS ester with an amino group of the protein to form an amide bond. Since several steps have to be performed after deposition of the SAM on the substrate, the yield reduces after each step, resulting in a lower yield of the immobilization degree.

Dojindo discloses succinimidyl alkane disulfide compounds such as dithiobis (succinimidyl octanoate) in generic forms without precise application data.

### SUMMARY OF THE INVENTION

The preferred embodiments provide a sensing device including a self-assembling monolayer suitable for the fabrication of a high selectivity, high stability and high reproducibility sensor, linked to a recognition molecule.

The preferred embodiments also provide a method for producing such a device.

The preferred embodiments also provide a compound suitable for forming the monolayer.

In a first embodiment, a device suitable for use in the preparation of a sensor is provided, the device including a substrate including a metal layer, the metal layer including a first region, wherein a first species is attached to the first region, the first species including a compound of chemical formula:

X—R₁—S—S—R₂—Y

wherein R₁ and R₂ independently include a spacer including n carbon atoms, wherein n includes an integer higher than 12, wherein X includes:

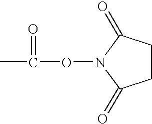

and wherein Y includes an organic group.

In an aspect of the first embodiment, R₁ and R₂ independently include a hydrocarbon chain.

In an aspect of the first embodiment, the hydrocarbon chain includes an alkane chain of formula (CH₂)ₙ.

In an aspect of the first embodiment, R₁ and R₂ independently include Q-R, wherein Q includes a hydrocarbon group, wherein Q is bound to a sulfur atom, and wherein R includes a chemical group for avoiding non-specific adsorption.

In an aspect of the first embodiment, R₁ and R₂ independently include (CH₂)ₐ—(CH₂—CH₂—O)_{b}—(CH₂)_{c}, wherein a includes an integer, b includes an integer, and c includes an integer.

In an aspect of the first embodiment, a includes an integer of from 1 to 20.

In an aspect of the first embodiment, b includes an integer of from 1 to 10.

In an aspect of the first embodiment, c includes an integer of from 1 to 3.

In an aspect of the first embodiment, n includes an integer of from 13 to 30.

In an aspect of the first embodiment, the spacer includes a heteroatom.

In an aspect of the first embodiment, R₁ and R₂ include a same chemical group.

In an aspect of the first embodiment, Y includes a chemical group selected from the group consisting of carboxyl, hydroxyl, cyano, amine, epoxy, and vinyl.

In an aspect of the first embodiment, Y includes:

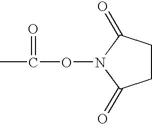

In an aspect of the first embodiment, the first species includes 16,16′-dithiohexadecanoic acid di(N-hydroxysuccinimide ester).

In an aspect of the first embodiment, the first species includes a compound of chemical formula:

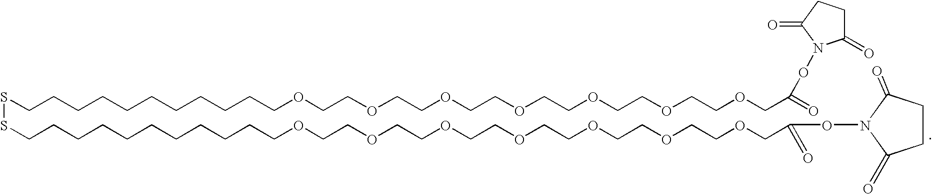

In an aspect of the first embodiment, the metal layer further includes a second region, wherein a second species is attached to the second region, wherein the second species a includes a compound of chemical formula:

W—R₃—S—S—R4—Z

wherein R₃ and R4 independently include a second spacer, W and Z independently include organic groups, and wherein the first species and the second species form a mixed self-assembled monolayer on the metal layer.

In an aspect of the first embodiment, the second spacer includes m carbon atoms interrupted by q heteroatoms, wherein q includes an integer greater than or equal to zero, wherein m includes an integer greater than zero, and wherein (m+q) includes an integer greater than 6.

In an aspect of the first embodiment, W and Z are independently selected from the group consisting of carboxyl, hydroxyl, cyano, amine, epoxy, and vinyl.

In an aspect of the first embodiment, the metal layer includes a metal selected from the group consisting of gold, silver, mercury, aluminum, platinum, palladium, copper, and alloys thereof.

In a second embodiment, a method for producing a device is provided, wherein the device is suitable for use in determining the presence of a target molecule, the method including the steps of providing a substrate including a metal layer; providing a first species including a compound of chemical formula:

X—R₁—S—S—R₂—Y

wherein R₁ and R₂ independently include a spacer including n carbon atoms, wherein n includes an integer greater than 12, wherein X includes:

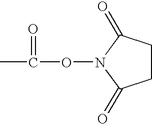

and wherein Y includes an organic group; and contacting the substrate to the first species, whereby a self-assembled monolayer is formed on the substrate.

In an aspect of the second embodiment, R₁ and R₂ independently include a hydrocarbon chain.

In an aspect of the second embodiment, the hydrocarbon chain includes an alkane chain of formula (CH₂)ₙ.

In an aspect of the second embodiment, R₁ and R₂ independently include Q—R, wherein Q includes a hydrocarbon group, wherein Q is bound to a sulfur atom, and wherein R includes a chemical group for avoiding non-specific adsorption.

In an aspect of the second embodiment, R₁ and R₂ independently include (CH₂)ₐ—(CH₂-CH₂—O)_{b}—(CH₂)_{c}, wherein a includes an integer, wherein b includes an integer, and wherein c includes an integer.

In an aspect of the second embodiment, the spacer includes a heteroatom.

In an aspect of the second embodiment, the method further includes the step of covalently binding a recognition molecule to X.

In an aspect of the second embodiment, the recognition molecule includes a chemical compound including a free NH₂ group.

In an aspect of the second embodiment, the recognition molecule is selected from the group consisting of antigens, antibodies, nucleic acid strands, hormones, enzymes, and polyaminoacids.

In an aspect of the second embodiment, the method further includes the steps of providing a second species, the second species including a compound different from the first species; and contacting the substrate with the second species, whereby a mixed self-assembling monolayer is formed.

In an aspect of the second embodiment, the second species includes a compound of chemical formula:

W—R₃—S—S—R₄—Z

wherein R₃ and R₄ independently include a second spacer, and wherein W and Z independently include an organic group.

In an aspect of the second embodiment, W and Z are independently selected from the group consisting of carboxyl, hydroxyl, cyano, amine, epoxy, and vinyl.

In a third embodiment, a compound is provided, wherein the compound is suitable for use in forming a monolayer on a sensor device, the compound including a formula:

X—R₁—S—S—R₂—Y

wherein R₁ and R₂ independently include a spacer including n carbon atoms, wherein n includes an integer greater than 12, wherein X includes:

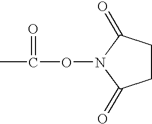

and wherein Y includes an organic group.

In an aspect of the third embodiment, R₁ and R₂ independently include a hydrocarbon chain.

In an aspect of the third embodiment, the hydrocarbon chain includes an alkane chain of a formula (CH₂)ₙ.

In an aspect of the third embodiment, R₁ and R₂ independently include Q-R, wherein Q includes a hydrocarbon group, wherein Q is bound to a sulfur atom, and wherein R includes a chemical group for avoiding non-specific adsorption.

In an aspect of the third embodiment, R₁ and R₂ independently include (CH₂)ₐ—(CH₂—CH₂—O)_{b}—(CH₂)_{c}, wherein a includes an integer, wherein b includes an integer, and wherein c includes an integer.

In an aspect of the third embodiment, a includes an integer of from 1 to 20.

In an aspect of the third embodiment, b includes an integer of from 1 to 10.

In an aspect of the third embodiment, c includes an integer of from 1 to 3.

In an aspect of the third embodiment, n includes an integer greater than 15.

In an aspect of the third embodiment, the spacer includes at least one heteroatom.

In an aspect of the third embodiment, R₁ and R₂ include a same chemical group.

In an aspect of the third embodiment, Y is selected from the group consisting of carboxyl, hydroxyl, cyano, amine, epoxy, and vinyl.

In an aspect of the third embodiment, X and Y include a same chemical group.

In an aspect of the third embodiment, the compound includes 16,16′-dithiohexadecanoic acid di(N-hydroxysuccinimide ester).

In an aspect of the third embodiment, the compound is of chemical formula:

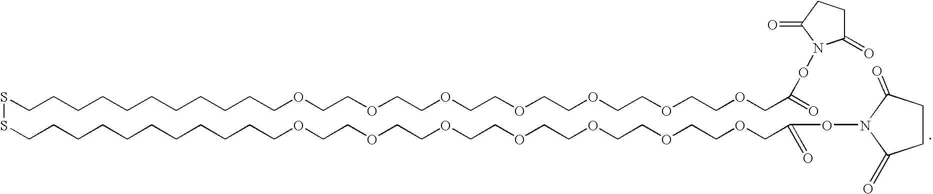

In a fourth embodiment, use of the compound of the first embodiment for the preparation of a self-assembling monolayer on a substrate of a sensor device is provided.

In a fifth embodiment, a sensor suitable for use in detecting an analyte is provided, the sensor including a compound including a formula:

X—R₁—S—S—R₂—Y

wherein R₁ and R₂ independently include a spacer including n carbon atoms, wherein n includes an integer greater than 12, wherein X includes:

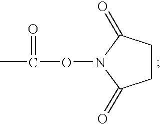

and wherein Y includes an organic group; and wherein a recognition molecule is covalently bonded to X.

In an aspect of the fifth embodiment, the recognition molecule is selected from the group consisting of antigens, antibodies, nucleic acid strands, hormones, enzymes, and polyaminoacids.

In an aspect of the fifth embodiment, the transducer is selected from the group consisting of surface plasmon resonance sensors, surface acoustic wave sensors, quartz crystal microbalances, amperometric sensors, capacitive sensors, interdigitated electrodes, and chemically modified field effect transistors.

In a sixth embodiment, a method of detecting an analyte is provided, the method including the steps of contacting a sensor with a sample including an analyte, the sensor including a transducer to which a compound is chemisorbed, the compound including a formula:

X—R₁—S—S—R₂—Y

wherein R₁ and R₂ independently include a spacer including n carbon atoms, wherein n includes an integer greater than 12, wherein X includes:

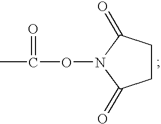

and wherein Y includes an organic group; and wherein a recognition molecule capable of recognizing the analyte is covalently bonded to X; and measuring an electrical signal via the transducer, wherein the electrical signal correlates with a concentration of the analyte in the sample.

### BRIEF DESCRIPTION OF THE DRAWINGS

FIG. 1 represents 16,16′-Dithiohexadecanoic acid di(N-hydroxysuccinimide ester) deposited on a substrate having a gold layer.

FIG. 2 represents a Grazing Angle Fourier Transform InfraRed (FTIR) spectrum of 16,16′-dithiohexadecanoic acid di(N-hydroxysuccinimide ester) deposited on a substrate having a gold layer.

FIGS. 3a, 3b, and 3c represent the molecules used for the comparative tests deposited on a substrate having a gold layer: a) 16-mercapto-1-hexadecanoic acid; b) 11-mercapto-1-undecanol and 16-mercapto-1-hexadecanoic acid; and c) 16-mercapto-1-hexadecanoic acid and 2-(2-(2-(6-mercapto hexyloxy)ethoxy)ethoxy)ethanol (6-polyethyleneoxide).

FIG. 4 represents the detected concentration of human transferrine for different types of monolayers versus RIU (Refractive Index Units).

FIG. 5 represents a mixed monolayer of 16,16′-dithiohexadecanoic acid di(N-hydroxysuccinimide ester) and 10,10′-dithioundecanol on a gold substrate.

FIG. 6 represents an interdigitated electrode configuration suitable for the fabrication of a sensor.

FIG. 7 represents the chemical structure of another chemical compound.

FIG. 8 represents chemical compounds to be used together with the molecule represented in FIG. 7 (mixed monolayers).

FIG. 9 represents anti-human transferrin immobilized on 16,16′-Dithiohexadecanoic acid di(N-hydroxysuccinimide ester). Experiments 1-4 refer to experiments performed at different times.

FIG. 10 represents 16,16′-Dithiohexadecanoic acid di(N-hydroxysuccinimide ester) reacting with antibodies (step 101) and blocked with a blocking agent (step 102).

FIG. 11 represents the recognition of a specific analyte HT and a non-specific analyte IgG on a surface with Streptavidin immobilization.

### DETAILED DESCRIPTION OF THE PREFERRED EMBODIMENT

The following description and examples illustrate a preferred embodiment of the present invention in detail. Those of skill in the art will recognize that there are numerous variations and modifications of this invention that are encompassed by its scope. Accordingly, the description of a preferred embodiment should not be deemed to limit the scope of the present invention.

In a first aspect of the preferred embodiments, a compound, suitable for the fabrication of a self-assembling monolayer, is provided. The compound has the chemical formula:

X—R₁—S—S—R₂—Y

wherein R₁ and R₂ represent independently from each other a spacer of n carbon atoms, wherein n is an integer higher than 11; wherein X represents:

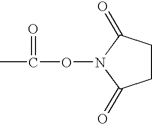

and Y represents an organic group. The compound allows the formation of a self-assembled monolayer.

The group:

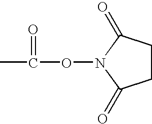

is the NHS, or N-hydroxysuccinimidyl, group.

Self-assembled monolayers are considered as a relative ordered assembly of molecules that spontaneously attach (or chemisorb) on a surface. The molecules are preferably oriented parallel and preferably under an angle of at least 45 degrees to the surface.

Each group being part of a self-assembling monolayer preferably contains a functional group for attaching to the surface (53) and a functional group that binds to the recognition molecule (35,55). In the preferred embodiments, the functional group being able to attach to the surface is the disulfide group —S—S— and the functional group being able to bind a recognition molecule is the NHS group.

The functional group —S—S— is able to adhere (chemisorb) to a surface such as a metal and can chemically interact with the metal surface (12, 52). The interaction between the sulfur atom and the substrate is known to people skilled in the art and is described in Nuzzo, R. G.; Allara, D. L.; J. Am. Chem. Soc. 1983, 105, 4481, and Abraham Ulman, An Introduction to Ultra thin Organic Films, Academic Press Inc, 1991.

The NHS group (55) can be used for surface immobilization of a recognition molecule. The recognition molecules can be bound to this group and in particular to the NH₂ group of a recognition molecule.

The NHS group is highly reactive towards thiol groups and a chemical compound including a thiol would react with the NHS group. Therefore, a disulfide is required such that binding between the NHS group and the sulfur atoms is prevented.

Y can be a chemical group selected from the group consisting of carboxyl, hydroxyl, cyano, amine, epoxy, and vinyl groups. In a preferred embodiment, Y is:

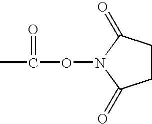

R₁ and R₂ represent independently from each other a spacer (13, 31, 32, 33, 34) of n carbon atoms, wherein n is an integer higher than 6 and preferably 11. The spacer promotes the formation of a self-assembling monolayer and can be a hydrocarbon chain. “Hydrocarbon” chain can be understood as including an alkyl, alkenyl, alkynyl, cyclic alkyl, aryl, alkyl bound to aryl, alkenyl bound to aryl, and alkynyl bound to aryl. In an embodiment, the spacer is an alkane. The spacer can also represent a hydrocarbon interrupted by a —CO— (ketone), —CONH, —CONHCO—, —CSNH—, —CS—, and the like. The hydrocarbon chain can also be branched. The heteroatom can be selected from the group consisting of —NH—, —O—, —S—, and —CS—. In particular, the heteroatom can be 0. The spacer can include a first part which is a hydrocarbon chain and a second part which is a hydrocarbon chain interrupted by a heteroatom such as oxygen. R₁ and R₂ can have the same chemical composition such that a symmetrical molecule is formed. Symmetrical molecules have generally the advantage of a more straightforward synthesis.

In a preferred embodiment, n is an integer. In a preferred embodiment n is higher than 4, higher than 6, higher than 8, higher than 10, higher than 11, higher than 12, higher than 13, higher than 15 or higher than 20. Alternatively, n is from 12 to 30, from 13 to 30, from 12 to 25, from 12 to 30, from 13 to 30, or from 12 to 22. Preferably, n is from 13 to 25. Most preferably, n is 16.

In a particular embodiment, R₁ and R₂ are independently from each other a spacer including two parts, a first part for obtaining a stable ordered monolayer and a second part for avoiding non-specific adsorption. In a particular embodiment, R₁ and R₂ are independently from each other (CH₂)ₐ—(CH₂—CH₂—O)_{b}—(CH₂)_{c}, a being an integer, b being an integer and c being an integer. The alkane chain is to achieve a stable ordered and reproducible system while the polyethyleneoxide groups are for avoiding non-specific adsorption. The variable “a” is preferably from 1 to 20, from 6 to 20, or from 6 to 15. The variable “b” is preferably from 1 to 10, and from 1 to 8. The variable “c” is an integer between 1 and 3. Preferably c can be 1, 2, or 3. The total number of carbon atoms n is preferably higher than 11, higher than 12, higher than 15, higher than 22, or higher than 25.

In a particularly preferred embodiment, the chemical compound is 16,16′-dithiohexadecanoic acid di(N-hydroxysuccinimide ester), with molecular formula C₂₇H₄₂N₂O₈S₂ and molecular weight: 586.

The chemical compound can be characterized as follows: mass spectroscopy: molecular weight: 769.12; H NMR: δ2.83 singlet, 2.68 triplet, 2.60 triplet, 1.78-1.63 multiplet, 1.43-1.29 multiplet.

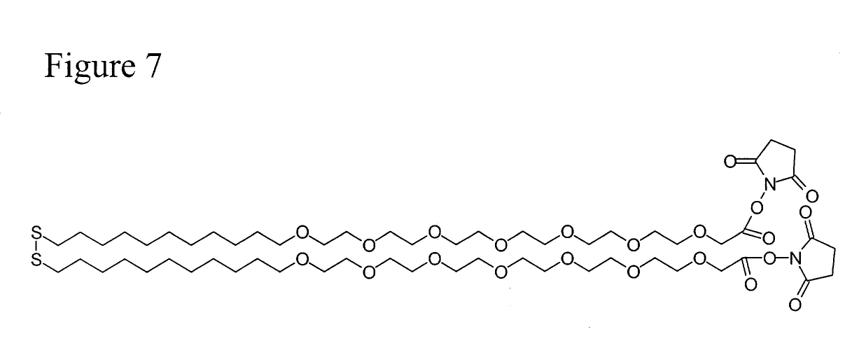

In another preferred embodiment, the chemical compound is the chemical compound as shown in FIG. 7 with the molecular formula C₅₈H₁₀₄N₂O₂₂S₂ and molecular weight: 1245,58.

In a second aspect of the preferred embodiments, a device, suitable for the fabrication of a sensor, is provided. The device includes: a substrate including a metal layer, the metal layer including at least two regions, wherein to a first region is attached a first species, the first species being the compound provided in the first aspect of the preferred embodiments, wherein to a second region is attached a second species having the chemical formula:

W—R₃—S—S—R₄-Z

wherein W and Z represent organic groups, R₃ and R₄ represent independently from each other spacer, optionally interrupted by a heteroatom. The first species and the second species are selected such that a mixed self-assembled monolayer is formed on the metal layer. A mixed self-assembled monolayer results in better sensitivity of the recognition molecule towards the target molecule in the medium. Certain species are used to prevent non-specific adsorption.

The molar ratio of the second species or the first species can be 1000:1, 500:1, 100:1, 80:1, 70:1, 60:1, 50:1, 20:1, 10:1, 5:1, 95:5, 90:10, 80:20, 70:30, or 60:40. The final ratio of the second and the first species can be determined by spectroscopic techniques available to a person skilled in the art.

A mixed monolayer is desired as it results in a better sensitivity of the recognition molecule to the target molecule in the medium. Non-specific adsorption is preferably avoided when the device is used as a sensor. Non-specific adsorption refers to interaction between the recognition molecule immobilized at the surface and any species being present in a medium that preferably contains the target molecule. “Any species” excludes the target molecule.

The second species can have the chemical formula W—R₃—S—S—R₄-Z, wherein R₃ and R₄ represent independently a spacer of m carbon atoms optionally interrupted by q heteroatoms and wherein m or (m+q) being an integer. Preferably, m or (m+q) are lower than n or (n+p).

The spacer promotes the formation of a self-assembling monolayer. The spacer can be a hydrocarbon chain. Hydrocarbon chain can be understood as including an alkyl, alkenyl, alkynyl, cyclic alkyl, aryl, alkyl bound to aryl, alkenyl bound to aryl, alkynyl bound to aryl and can also represent a hydrocarbon interrupted by a —CO—(ketone), —CONH, —CONHCO—, —CSNH—, —CS—, and the like. The hydrocarbon chain can be branched. The heteroatom can be selected from the group consisting of —NH—, —O—, —S—, and —CS—. R₃ and R₄ can have the same chemical composition such that a symmetrical molecule is formed. Symmetrical molecules generally have the advantage of a more straightforward synthesis.

In an embodiment, R₃ and R₄ are independently a spacer of m carbon atoms, optionally interrupted by q heteroatoms wherein (m+q) or m is an integer higher than 6. In a preferred embodiment, R₃ and R₄ are independently selected from the group consisting of an alkyl chain (CH₂)ₘ (81) with m an integer higher than 6 and an alkyl chain interrupted by q heteroatom with (m+q) higher than 6. W and Z are independently selected from the group consisting of carboxyl, hydroxyl, cyano, amine, epoxy, and vinyl groups.

In another embodiment, R₃ and R₄ are independently from each other a spacer including two parts, a first part for obtaining a stable ordered monolayer (82) and a second part for avoiding non-specific adsorption (83). In a particular embodiment, R₃ and R4 are independently from each other (CH₂)ₑ—(CH₂—CH₂—O)_{f}(CH₂)_{g}, e being an integer, f being an integer and g being an integer. The alkane chain is to achieve a stable ordered and reproducible system while the polyethyleneoxide groups are for avoiding non-specific adsorption. The variable “e” is preferably an integer from 1 to 20, from 5 to 20, from 5 to 15, from 5 to 12, 6, or 11. The variable “f” is preferably an integer from 1 to 10, from 1 to 8, from 2 to 6, 3, 4, or 5. The variable “g” is an integer preferably from 1 to 3. Preferably, g is 1, 2, or 3. The total number of carbon atoms n is preferably higher than 3, higher than 6, higher than 8, or higher than 10.

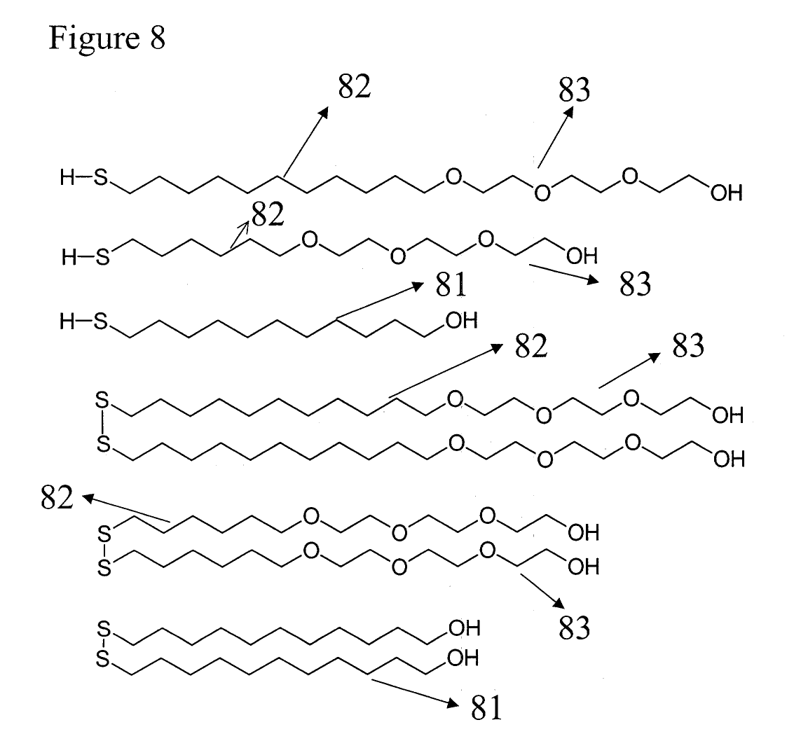

Examples of the second species are represented in FIG. 8.

In a preferred embodiment, disulfides are used, such that the reaction between the sulfur atoms of the second species and the NHS group of the first species is avoided during the deposition of the mixed monolayer.

W and Z are organic groups selected such that non-specific adsorption is reduced. The groups W and Z preferably have a functionality that does not adhere to the target molecule in the medium and that does not attach to the surface. Particularly, W and Z can be OH or a sugar moiety.

In a preferred embodiment, the second species has the chemical formula HO—(CH₂)ₘ—S—S—(CH₂)ₘ—OH, m being an integer higher than 4, higher than 7, preferably m being equal to 10.

In another particular embodiment, the molecules as referred to in FIG. 8 are employed.

The first species and the second species can be selected such that they can be deposited from a solution or a mist on the required (first or second) part of the substrate. The substrate preferably contains a metal layer such as, but not limited hereto, gold, silver, mercury, aluminum, platinum, palladium, copper, cadmium, lead, iron, chromium, manganese, tungsten and alloys thereof.

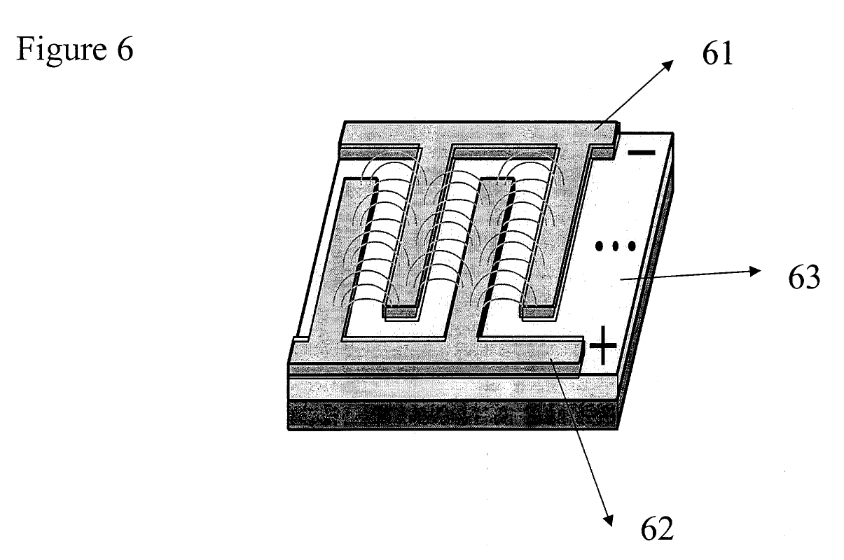

According to one of the preferred embodiments, a combination of gold as surface material and the first and second species as described herein is selected. The metal layer can, but does not necessarily, cover the whole substrate. The metal layer can be finger-shaped such as interdigitated electrodes (see FIG. 6).

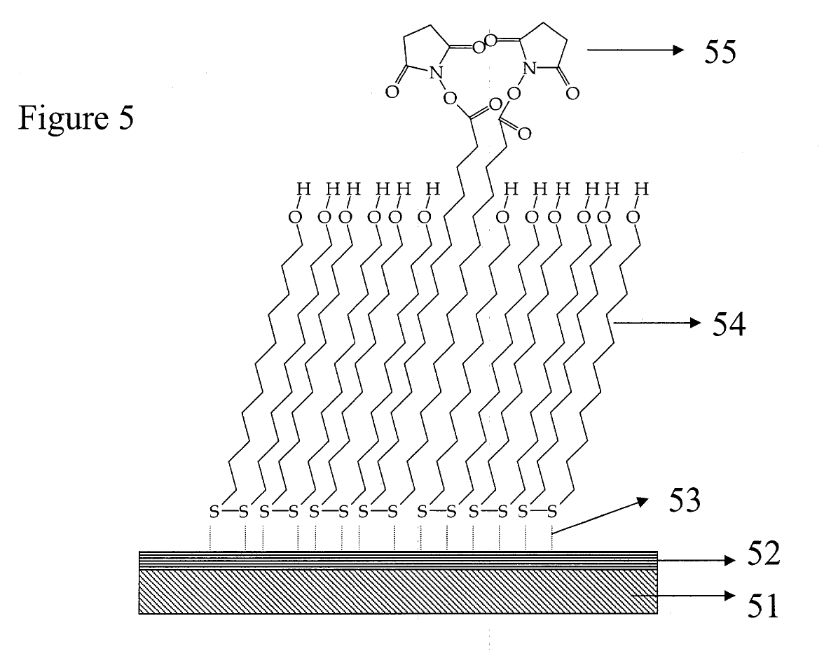

In another preferred embodiment, a device as represented in FIG. 5 is provided. The device includes a substrate having a gold surface. A mixed monolayer is chemisorbed (53) to the gold layer (52) deposited on a substrate (51). The mixed monolayer includes two species, 16,16′-dithiohexadecanoic acid di(N-hydroxysuccinimide ester) (55) and HO—(CH₂)₁₁—S—S—(CH₂)₁₁—OH (54) in a 70:30 ratio.

The NHS group in the first species as described in the first aspect of the preferred embodiments is able to bind a NH₂— group of the recognition molecule (NH₂—B) such that a O═C—NH—R group is formed.

Preferably, the first species and the second species are dissolved in the same solvent and both species are subjected together to the substrate. The solvent is preferably essentially free of water.

In second aspect of the preferred embodiments, a device, suitable for the fabrication of a sensor is provided. The device includes: a substrate including a metal layer, the metal layer including at least a first region wherein to a first region is attached a first species having the chemical formula:

X—R₁—S—S—R₂—Y

wherein R₁ and R₂ represent independently from each other a spacer of n carbon atoms, wherein n is an integer higher than 12; wherein X represents:

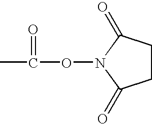

and Y represents an organic group. The first species can be characterized by the properties described in the first aspect of the preferred embodiments.

In an embodiment, the metal layer of the device further includes a second region, wherein to the second region is attached a second species having the chemical formula:

W—R₃—S—S—R₄-Z,

wherein R₃ and R₄ represent independently from each a spacer, optionally interrupted by a heteroatom, W and Z being organic groups, and wherein the first species and the second species forms a mixed self-assembled monolayer on the metal layer. The second species can be characterized by the properties described in the first aspect of the preferred embodiments.

The metal is selected from the groups consisting of gold, silver, mercury, aluminum, platinum, palladium, copper, or alloys thereof.

In a third aspect of the preferred embodiments, a sensor is provided. The chemical nature of the self assembling monolayer used in the sensor is different of the one used in the device in such a way that the chemical group of the SAM has a sensing or a recognition function towards a chemical compound belonging to the external medium (target molecule to be analyzed) the sensor includes: a substrate including a metal layer, the metal layer including at least two regions, wherein to a first region is attached a fifth species, the fifth species having the chemical formula:

Y—R₁—S—S—R₂—CO—NH—B

wherein B is part of a recognition molecule NH₂—B.

NH₂—B is a recognition molecule, i.e., a molecule able to selectively interact with a target molecule that is present in an external medium. The recognition molecule can be, but is not limited hereto, a molecule, wherein the —NH₂ group is covalently bound to the C═O group of the NHS of the monolayer, already deposited on the substrate. The recognition molecule can be, but is not limited hereto, a nucleic acid strand (DNA, PNA, RNA), hormones, antibiotics, antibodies, antigens, enzymes, drugs or drugs of abuse or molecules such as recognition molecules for gases or ions.

Besides recognition molecules with a terminal —NH₂ group, other groups can be bound to the succinimide group. This surface synthesis requires an extra step, wherein a cross-linker molecule is bound to the NHS group and the recognition molecule.

According to a preferred embodiment, the sensor is suitable for determining the presence of a compound, such as a target molecule in a medium. Target molecule is understood as a chemical compound that is able to interact with the recognition molecule. The target molecule can be, but is not limited hereto, complementary nucleic acid strand (DNA, PNA, RNA), hormones, antibiotics, antibodies, antigens, enzymes, drugs or drugs of abuse or molecules such as specific molecules present in for gases or ions. The sensor can be arranged such that it acts as a biosensor chip (Surface Plasmon Resonance (SPR) chip, Surface Acoustic Wave (SAW) chip, and the like).

The device and the sensor can further include a transducer. The self-assembling monolayer is deposited on the metal surface of the transducer. The transducer can be part of, but is not limited hereto, a surface plasmon resonance sensor, surface acoustic wave sensors, quartz crystal microbalance, amperometric sensors, capacitive sensors, interdigitated electrodes, or chemically modified field effect transistors (ChemFETs). FIG. 6 represents a sensor including interdigitated electrodes as transducer, wherein 61 is the negative electrode, 62 is the positive electrode and 63 is a substrate on which the electrodes are deposited. The monolayer as described herein can be deposited on the electrodes.

In a fourth aspect of the preferred embodiments, a method for producing a device, suitable for determining the presence of a compound is provided. The method includes the steps of: providing a substrate including at least a metal layer, providing a first species having the formula:

X—R₁—S—S—R₂—Y

wherein R₁ and R₂ represent independently from each other a spacer of n carbons, wherein n is an integer higher than 11 or higher than 12, wherein X represents:

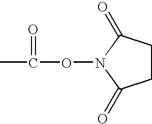

and wherein Y is an organic group, and subjecting the substrate to the first species to form a self-assembled monolayer on the substrate. The spacer of n carbon atoms can optionally being interrupted by p heteroatoms.

The first species can have the characteristics as described in the first and second aspect of the preferred embodiments.

Contrary to the prior art, the NHS terminated molecule can be directly deposited on the substrate without any intermediate step. This results in a higher yield.

The method can further include the step of covalently binding a recognition molecule NH₂—B to the X group of the self-assembling monolayer. The NH2 group of NH₂—B will react with the NHS group of the first species such that a CO—NH—B group is formed. In further step, substrate can be subjected to a blocking agent such that the unreacted NHS groups are deactivated. Consequently, interaction between the target molecule and the unreacted NHS groups are substantially avoided.

In a preferred embodiment, the method further includes the steps of providing a second species, the second species being different from the first species, and subjecting the substrate to the second species such that a mixed self-assembling monolayer is formed on the metal layer.

The second species can have the chemical formula and function as described in the first, second or third aspects of the preferred embodiments.

Preferably, the step of subjecting the substrate to a first species and subjecting the substrate to a second species are performed together, i.e. that the first species and the second species are dissolved in the same solvent and that both species are subjected together to the substrate. The solvent is preferably essentially free of water.

The method can further include the step of covalently binding a recognition molecule NH₂—B to the X group of the self-assembling monolayer. The NH2 group of NH₂—B will react with the NHS group of the first species such that a CO—NH—B group is formed. The interaction between the W and Z group of the second species and the recognition molecule is preferably as low as possible, there is preferably no covalent binding between the second species and the recognition molecule.

The method as described in the fourth aspect of the preferred embodiments results in an immobilization of the recognition molecule on the self-assembling monolayer which is high. This means that there are a lot of recognition sites such that the recognition can be higher and the detection limit can be lower. The value of the immobilization degree depends on the recognition molecule. For example, the immobilization degree for proteins is preferably higher than 3500 pg/mm², even more preferably higher than 4000 pg/mm². The high degree of immobilization of the recognition molecule can be obtained because the succinimide-terminated species is directly deposited on the substrate and thereafter, the amino groups of the recognition molecule can directly be coupled to the succinimide groups without an extra activation step.

Introducing an extra activation step (as mentioned in the prior art) lowers the yield of immobilization degree.

A mixed monolayer is desired as it results in a better sensitivity of the recognition molecule to the target molecule in the medium.

Experimental Results

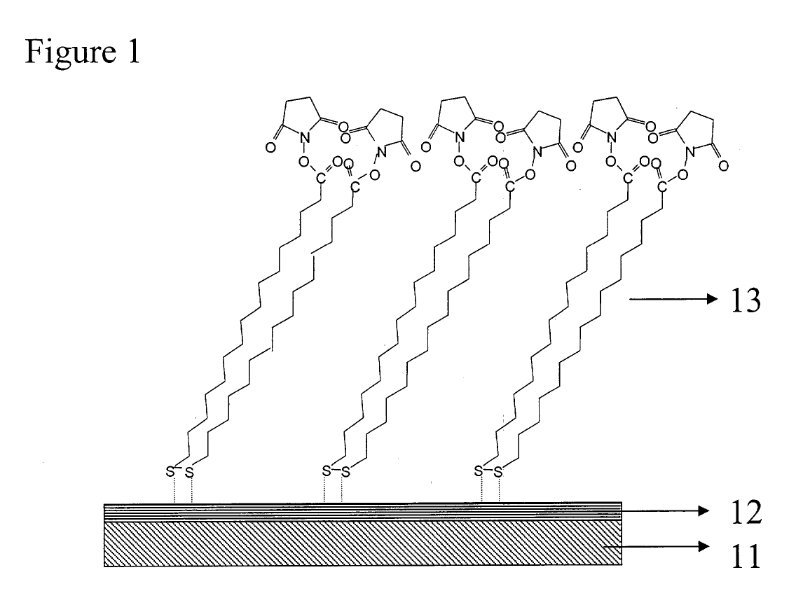

16,16′-Dithiohexadecanoic acid di(N-hydroxysuccinimide ester) (hereafter called DSH SAM) is deposited on a gold layer (12) of a substrate (11). The deposited molecule (13) is represented in FIG. 1.

The molecule is deposited from a water-free organic solvent like for example tetrahydrofuran (THF). The metal substrates are deposited in this solution and the optimal time (at least 3h) is used to organize the thiols into a self-assembled monolayer (SAM). Afterwards the substrate with the SAM is rinsed with THF and dried with nitrogen. Next step is putting this substrate in a solution with the recognition molecules. The recognition molecules will covalently bind without any activation step.

The synthesis of the molecule is as follows:

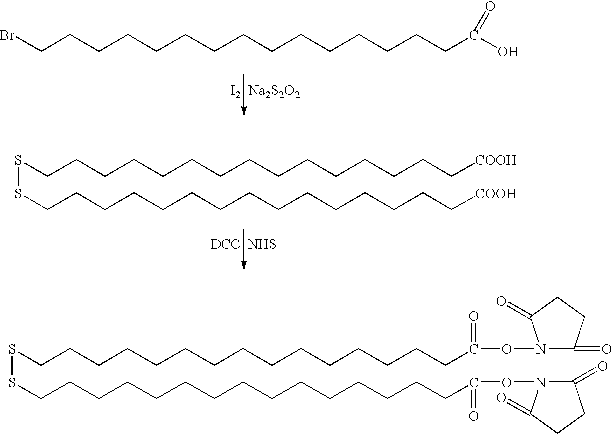

The contact angle of this molecule on-a gold surface is 43±1°. This is rather hydrophilic, which gives an indication that this surface is suitable for the immobilization of receptor proteins and that the qualities against non-specific interactions are normally acceptable.

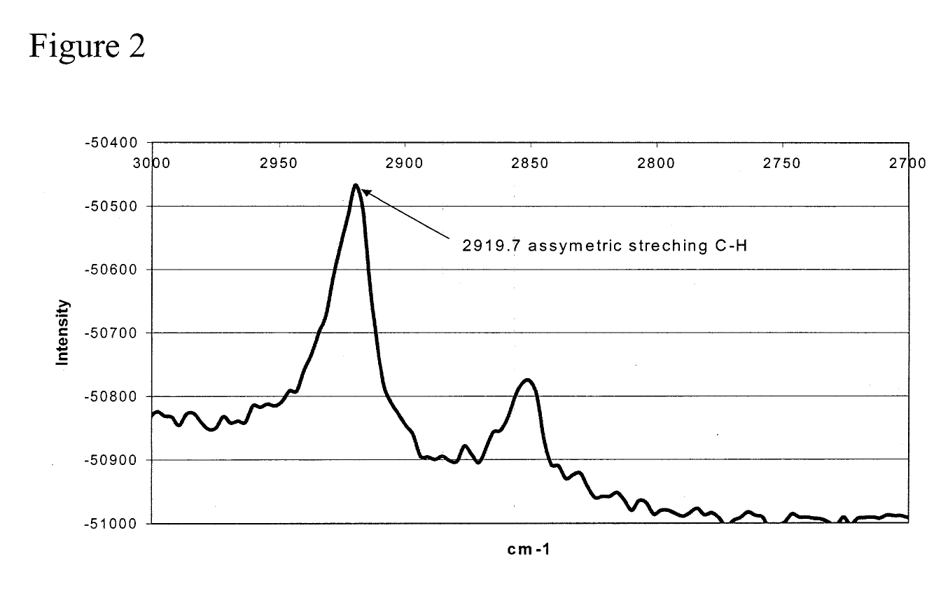

With Grazing Angle FTIR, we reveal that the packing of this molecule on gold is rather good despite the bulky groups and the disulfide bounds (see FIG. 2).

The asymmetric stretching at 2919.7 gives an indication on the packing of the monolayer. A perfect crystalline like monolayer has this peak at 2918 cm⁻¹ while spaghetti like structure would be around 2925 cm⁻¹. This monolayer can therefore be assigned as a rather well packed monolayer.

For DSH SAM, the deposited monolayer is well formed and can be used to attach antibodies on the surface. No activation step is necessary. The yield is much higher than on a normal thiol.

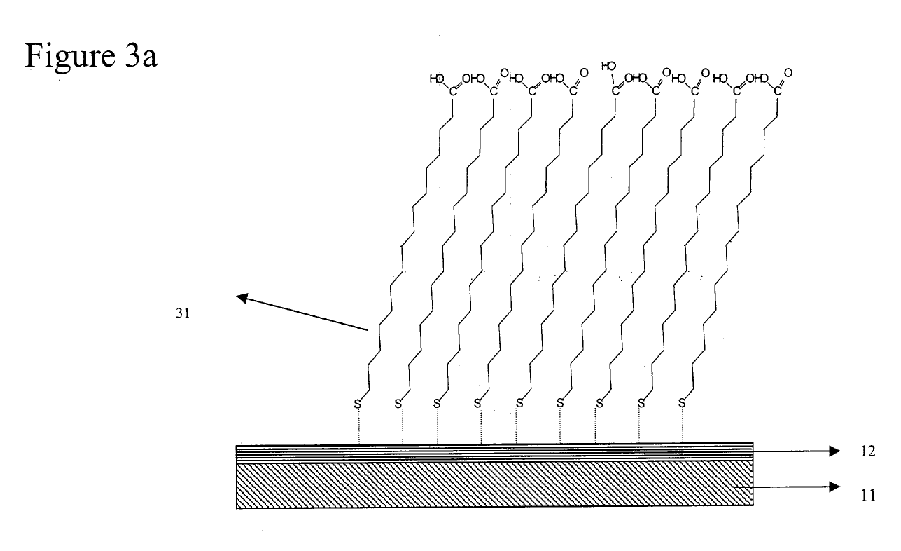

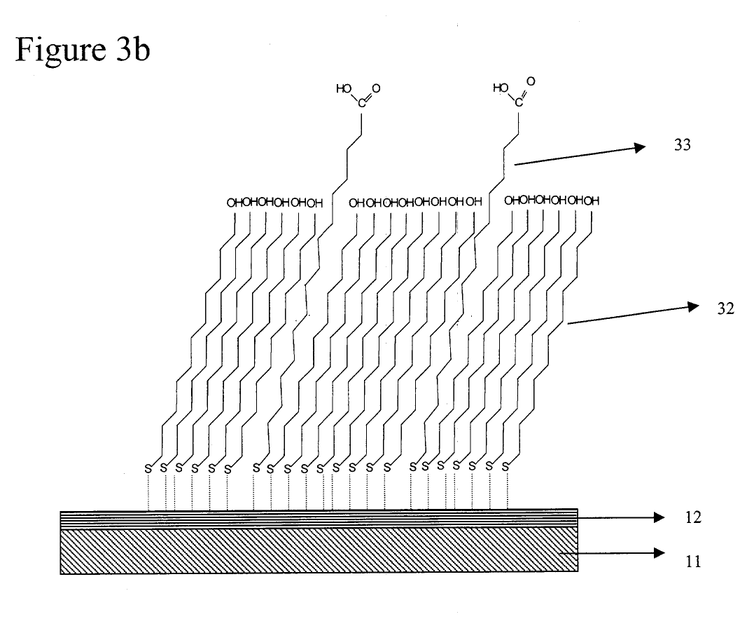

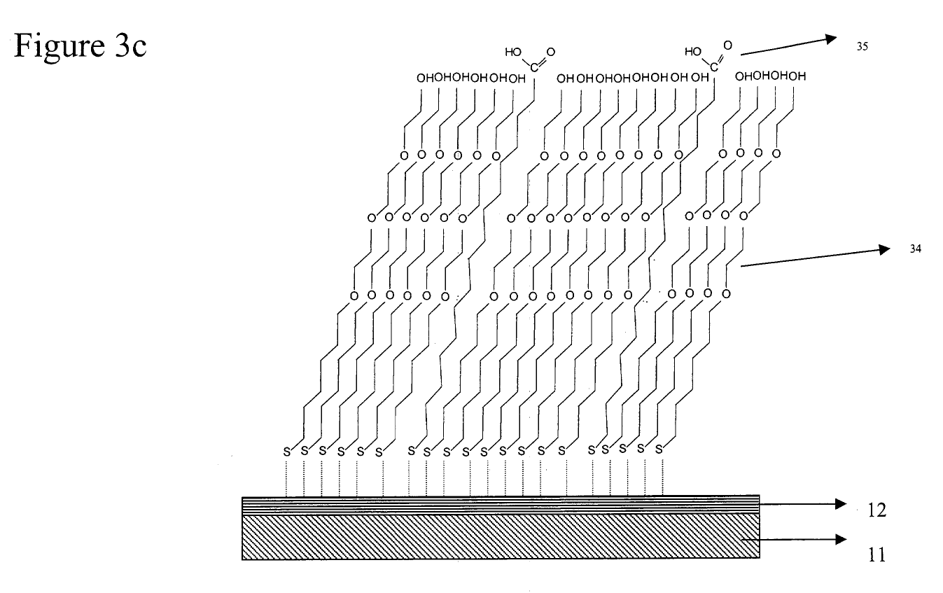

Comparative test between the molecule represented in FIG. 1 and the molecule represented in FIGS. 3a-c are performed with regard to the immobilization of antibodies on the molecules represented in FIGS. 3a-c.

FIG. 3a: a self-assembling monolayer of molecules 31 (deposited on a substrate 11 having a gold layer 12) is converted to a self-assembling monolayer including a N-hydroxysuccinimidyl (NHS) ester from the carboxylic acid groups of the self-assembling monolayer. In a next step, the ester is coupled to the free amine group of the protein (anti-human transferrine). The immobilization degree is 130 ng/cm² to 200 ng/cm².

FIG. 3b: a mixed self-assembling monolayer of molecules 32 and 33 is formed on a substrate 11 having a gold layer 12. The carboxylic acid groups (33) of the self-assembling monolayer are converted to a self-assembling monolayer including a N-hydroxysuccinimidyl (NHS) ester. In a next step, the ester is coupled to the free amine group of the protein (anti-human transferrine). The immobilization degree is 130 ng/cm² to 250 ng/cm².

FIG. 3c: A mixed Self-assembling monolayer of molecules 34 and 35 is formed on a substrate 11 having a gold layer 12. The self-assembling monolayer including carboxylic acid groups (35) is converted to a self-assembling monolayer including a N-hydroxysuccinimidyl (NHS) ester. In a next step, the ester is coupled to the free amine group of the protein (anti-human transferrine). The immobilization degree is preferably 130 ng/cm² to 200 ng/cm².

The immobilization of antibodies on DSH SAM (as represented in FIG. 1) resulted in >4383±13 pg/mm². It is clear that the immobilization degree is lower for the molecules represented in FIG. 3a-c compared to the molecule represented in FIG. 1.

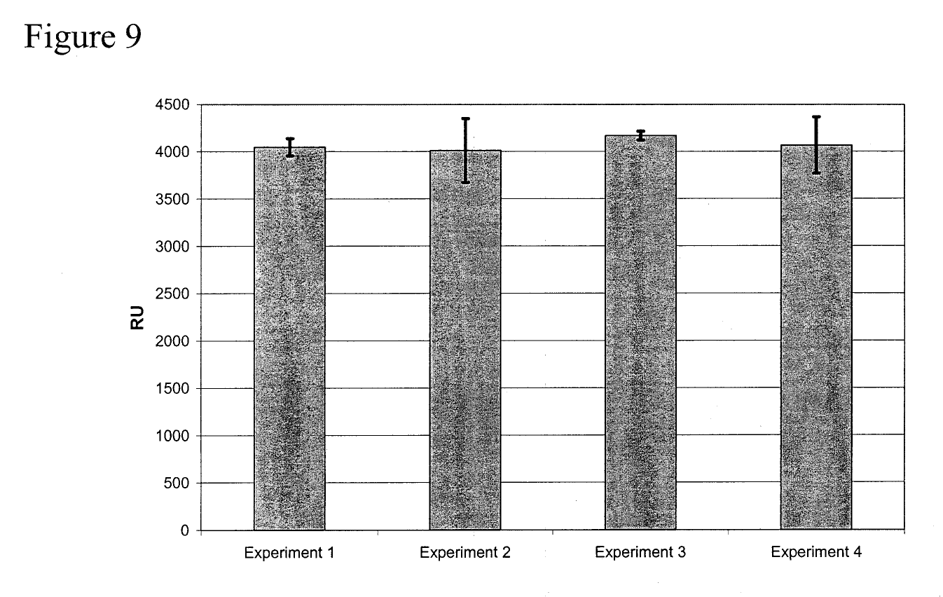

Anti-human Transferrin is immobilized on the DSH SAM, but different measurements were performed on different times and the solutions were prepared at different times. FIG. 9 shows very reproducible anti-HT immobilization. The difference between the different measurements was less than 3.75 %.

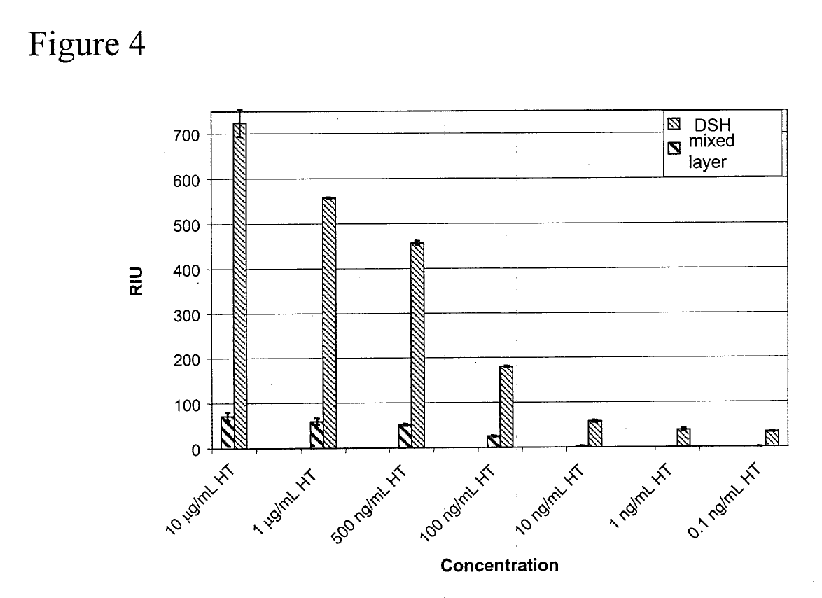

In a further experiment, the recognition of the antigen by the antibody is investigated. In this particular case, we immobilized anti-human transferrine (recognition molecule) and detected human transferrine (target molecule) in different concentrations. This is shown in FIG. 4. DSH SAM is compared to a mixed monolayer of 16-mercapto-1-hexadecanoic acid and 11-mercapto-1-undecanol, which showed the best results of the monolayers shown in FIG. 4.

The response is much higher with the DSH SAM compared to a mixed monolayer. In addition, the detection limit is also much lower.

In a next experiment, a mixed monolayer of DSH SAMs and a second species is formed, as shown in FIG. 5. In a mixed SAM, the DSH acts as the receptor for a recognition molecule, while the other thiols can have groups, which are good against non-specific adsorption like OH, CH₂CH₂OH, CH₂CH₂OCH₃, COOH, and the like.

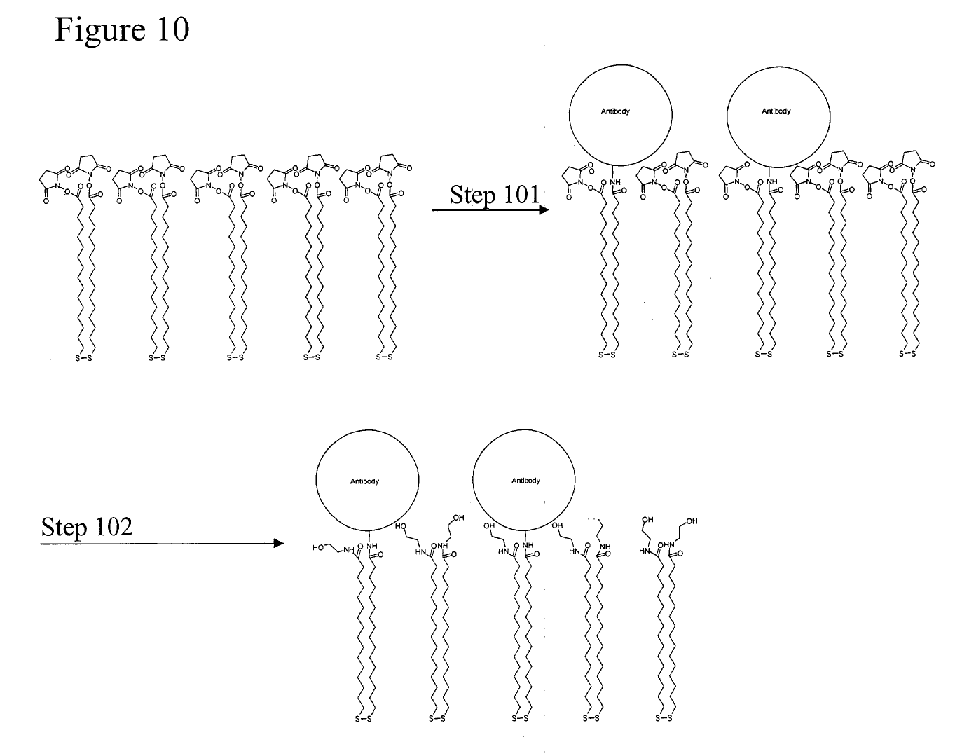

The recognition molecule can directly bind to the N-hydroxysuccinimide group or via a crosslinker to another group than NH₂ of the recognition molecule (step 101). Subsequently, the nonreacted NHS groups are preferably deactivated so that the analyte (antigen) reacts with the immobilized antibody and not with the surface (step 102). See FIG. 10. This is performed with ethanolamine. In this experiment, we replaced ethanolamine by a polyethyleneoxide (PEO) containing blocking. The PEO containing blocking agent is H₂N—(CH₂—CH₂—O)₃—H.

It was observed that there was no difference in antibody immobilization. There was no difference in recognition of the antigen (mansferrin) (data not shown).

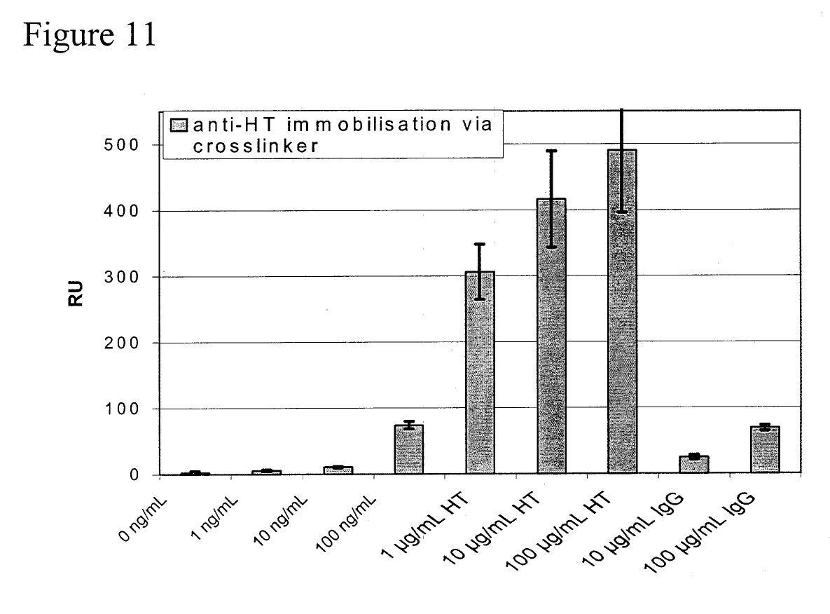

In a further experiment, A versatile surface is realized using an additional crosslinker containing PEO groups and biotin. After DSH deposition, a NHS terminated surface is achieved. Subsequently, the substrate is subjected to NH₂-polyethyleneoxide-biotin crosslinker. The amino group reacts with the NHS groups of the DSH surface. Subsequently, Streptavidin is immobilized, followed by biotinylated anti-HT. A surface that is sensitive to biotin-conjugated proteins or DNA is realized. In addition the PEO groups preferably prevent the non-specific adsorption. The Streptavidin immobilization on this surface is the recognition of a specific analyte HT and a non-specific analyte IgG are represented in FIG. 11. It is observed that the recognition of a specific analyte HT is much higher than the recognition of the non-specific analyte IgG.

In another experiment, the molecules as represented in FIG. 7 (hereafter called PEO SAM) are deposited on a gold layer (12) of a substrate (11).

The synthesis of the molecule was as follows. The starting molecule (to synthesize the above-mentioned molecule) was synthesized according to J. Lahiri, L. Isaacs, J. Toe, and G. M. Whitesides, Analytical Chemistry, 1999, 71, 777.

The starting molecule is as follows:

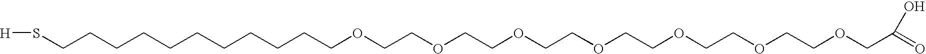

The synthesis route is as follows:

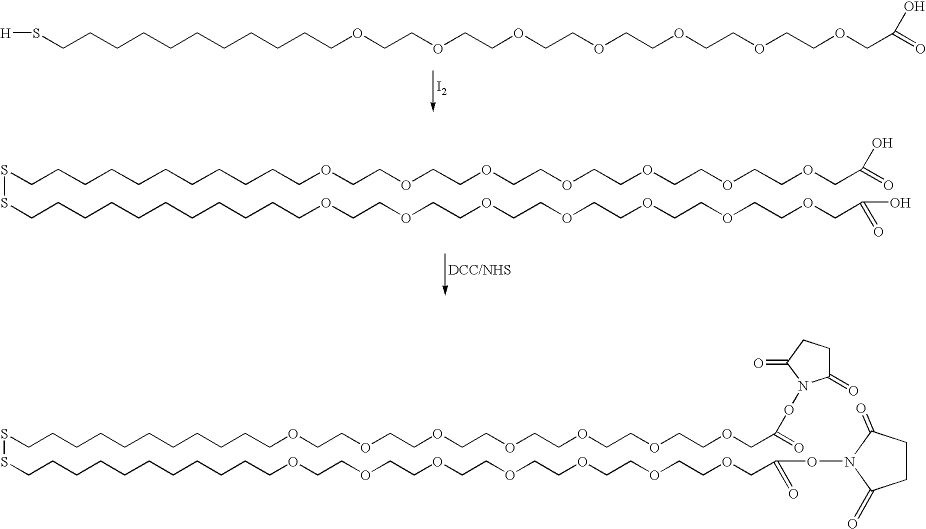

In another embodiment, mixed monolayers are deposited. Therefore, the above-mentioned molecules are deposited together with a molecule mentioned in FIG. 8. Mixed SAMs can have some additional advantages such as avoiding non-specific adsorption, avoiding steric hindrance, and enhanced sensitivity.

The enhanced qualities of the preactivated DSH SAMs can be used in combination with mixed SAMs.

The above description provides several methods and materials of the present invention. This invention is susceptible to modifications in the methods and materials, as well as alterations in the fabrication methods and equipment. Such modifications will become apparent to those skilled in the art from a consideration of this disclosure or practice of the invention provided herein. Consequently, it is not intended that this invention be limited to the specific embodiments provided herein, but that it cover all modifications and alternatives coming within the true scope and spirit of the-invention as embodied in the attached claims. All references cited herein are hereby incorporated by reference in their entireties.

What is claimed is:

1. A device suitable for use in the preparation of a sensor, the device comprising a substrate comprising a metal layer, the metal layer comprising a first region, wherein a first species is attached to the first region, the first species comprising a compound of chemical formula:
X—R₁—S—S—R₂—Y
wherein R₁ and R₂ independently comprise a spacer comprising n carbon atoms, wherein n comprises an integer higher than 12, wherein X comprises: 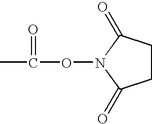
and wherein Y comprises an organic group.

2. The device of claim 1, wherein R₁ and R₂ independently comprise a hydrocarbon chain.

3. The device of claim 2, wherein the hydrocarbon chain comprises an alkane chain of formula (CH₂)ₙ.

4. The device of claim 1, wherein R₁ and R₂ independently comprise Q-R, wherein Q comprises a hydrocarbon group, wherein Q is bound to a sulfur atom, and wherein R comprises a chemical group for avoiding non-specific adsorption.

5. The device of claim 1, wherein R₁ and R₂ independently comprise (CH₂)ₐ—(CH₂—CH₂—O)_{b}—(CH₂)_{c}, wherein a comprises an integer, b comprises an integer, and c comprises an integer.

6. The device of claim 5, wherein a comprises an integer of from 1 to 20.

7. The device of claim 5, wherein b comprises an integer of from 1 to 10.

8. The device of claim 5, wherein c comprises an integer of from 1 to 3.

9. The device of claim 1, wherein n comprises an integer of from 13 to 30.

10. The device of claim 1, wherein the spacer comprises a heteroatom.

11. The device of claim 1, wherein R₁ and R₂ comprise a same chemical group.

12. The device of claim 1, wherein Y comprises a chemical group selected from the group consisting of carboxyl, hydroxyl, cyano, amine, epoxy, and vinyl.

13. The device of claim 1, wherein Y comprises:

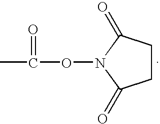

14. The device of claim 1, wherein the first species comprises 16,16′-dithiohexadecanoic acid di(N-hydroxysuccinimide ester).

15. The device of claim 1 wherein the first species comprises a compound of chemical formula:

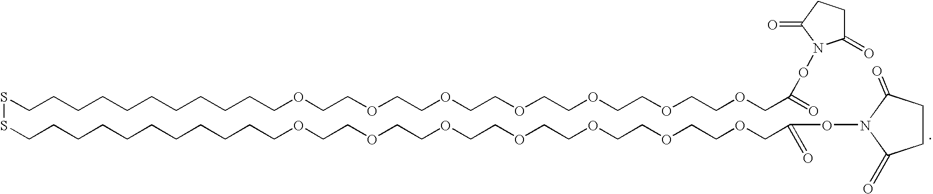

16. The device of claim 1, wherein the metal layer further comprises a second region, wherein a second species is attached to the second region, wherein the second species a comprises a compound of chemical formula:
W—R₃—S—S—R₄-Z
wherein R₃ and R₄ independently comprise a second spacer, W and Z independently comprise organic groups, and wherein the first species and the second species form a mixed self-assembled monolayer on the metal layer.

17. The device of claim 16, wherein the second spacer comprises m carbon atoms interrupted by q heteroatoms, wherein q comprises an integer greater than or equal to zero, wherein m comprises an integer greater than zero, and wherein (m+q) comprises an integer greater than 6.

18. The device of claim 16, wherein W and Z are independently selected from the group consisting of carboxyl, hydroxyl, cyano, amine, epoxy, and vinyl.

19. The device of claim 1, wherein the metal layer comprises a metal selected from the group consisting of gold, silver, mercury, aluminum, platinum, palladium, copper, and alloys thereof.

20. A method for producing a device, wherein the device is suitable for use in determining the presence of a target molecule, the method comprising the steps of:
providing a substrate comprising a metal layer;
providing a first species comprising a compound of chemical formula:
X—R₁—S—S—R₂—Y
wherein R₁ and R₂ independently comprise a spacer comprising n carbon atoms, wherein n comprises an integer greater than 12, wherein X comprises: 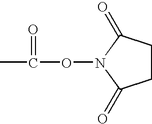
and wherein Y comprises an organic group; and
contacting the substrate to the first species, whereby a self-assembled monolayer is formed on the substrate.

21. The method of claim 20, wherein R₁ and R₂ independently comprise a hydrocarbon chain.

22. The method of claim 20, wherein the hydrocarbon chain comprises an alkane chain of formula (CH₂)ₙ.

23. The method of claim 20, wherein R₁ and R₂ independently comprise Q-R, wherein Q comprises a hydrocarbon group, wherein Q is bound to a sulfur atom, and wherein R comprises a chemical group for avoiding non-specific adsorption.

24. The method of claim 20, wherein R₁ and R₂ independently comprise (CH₂)ₐ—(CH₂—CH₂—O)_{b}—(CH₂)_{c}, wherein a comprises an integer, wherein b comprises an integer, and wherein c comprises an integer.

25. The method of claim 20, wherein the spacer comprises a heteroatom.

26. The method of claim 20, further comprising the step of covalently binding a recognition molecule to X.

27. The method of claim 26, wherein the recognition molecule comprises a chemical compound comprising a free NH₂ group.

28. The method of claim 26, wherein the recognition molecule is selected from the group consisting of antigens, antibodies, nucleic acid strands, hormones, enzymes, and polyaminoacids.

29. The method of claim 20, further comprising the steps of:
providing a second species, the second species comprising a compound different from the first species; and
contacting the substrate with the second species, whereby a mixed self-assembling monolayer is formed.

30. The method of claim 29, wherein the second species comprises a compound of chemical formula:
W—R₃—S—S—R₄-Z
wherein R₃ and R₄ independently comprise a second spacer, and wherein W and Z independently comprise an organic group.

31. The method of claim 30, wherein W and Z are independently selected from the group consisting of carboxyl, hydroxyl, cyano, amine, epoxy, and vinyl.

32. A compound, wherein the compound is suitable for use in forming a monolayer on a sensor device, and wherein the compound is of chemical formula:
X—R₁—S—S—R₂—Y
wherein R₁ and R₂ independently comprise a spacer comprising n carbon atoms, wherein n comprises an integer greater than 12, wherein X comprises: 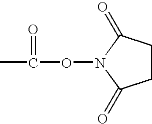
and wherein Y comprises an organic group.

33. The compound of claim 32, wherein R₁ and R₂ independently comprise a hydrocarbon chain.

34. The compound of claim 32, wherein the hydrocarbon chain comprises an alkane chain of a formula (CH₂)ₙ.

35. The compound of claim 32, wherein R₁ and R₂ independently comprise Q-R, wherein Q comprises a hydrocarbon group, wherein Q is bound to a sulfur atom, and wherein R comprises a chemical group for avoiding non-specific adsorption.

36. The compound of claim 32, wherein R₁ and R₂ independently comprise (CH₂)ₐ—(CH₂—CH₂—O)_{b}—(CH₂)_{c}, wherein a comprises an integer, wherein b comprises an integer, and wherein c comprises an integer.

37. The compound of claim 36, wherein a comprises an integer of from 1 to 20.

38. The compound of claim 36, wherein b comprises an integer of from 1 to 10.

39. The compound of claim 36, wherein c comprises an integer of from 1 to 3.

40. The compound of claim 32, wherein n comprises an integer greater than 15.

41. The compound of claim 32, wherein the spacer comprises at least one heteroatom.

42. The compound of claim 32, wherein R₁ and R₂ comprise a same chemical group.

43. The compound of claim 32, wherein Y is selected from the group consisting of carboxyl, hydroxyl, cyano, amine, epoxy, and vinyl.

44. The compound of claim 32, wherein X and Y comprise a same chemical group.

45. The compound of claim 32, wherein the compound comprises 16,16′-dithiohexadecanoic acid di(N-hydroxysuccinimide ester).

46. The compound of claim 32, wherein the compound is of chemical formula:

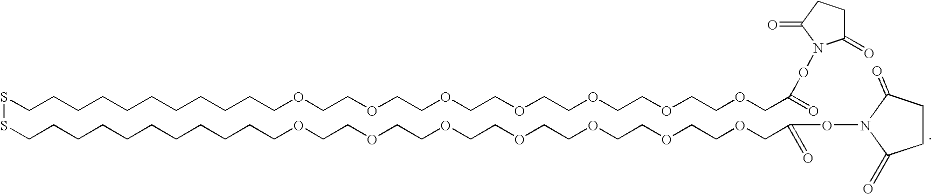

47. Use of the compound of claim 32 for the preparation of a self-assembling monolayer on a substrate of a sensor device.

48. A sensor suitable for use in detecting an analyte, the sensor comprising a compound of chemical formula:
X—R₁—S—S—R₂—Y
wherein R₁ and R₂ independently comprise a spacer comprising n carbon atoms, wherein n comprises an integer greater than 12, wherein X comprises: 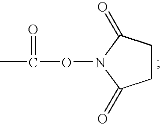
and wherein Y comprises an organic group; and wherein a recognition molecule is covalently bonded to X.

49. The sensor of claim 48, wherein the recognition molecule is selected from the group consisting of antigens, antibodies, nucleic acid strands, hormones, enzymes, and polyaminoacids.

50. The sensor of claim 48, wherein the transducer is selected from the group consisting of surface plasmon resonance sensors, surface acoustic wave sensors, quartz crystal microbalances, amperometric sensors, capacitive sensors, interdigitated electrodes, and chemically modified field effect transistors.

51. A method-of detecting an analyte, the method comprising the steps of:
contacting a sensor with a sample comprising an analyte, the sensor comprising a transducer to which a compound is chemisorbed, wherein the compound is of chemical formula:
X—R₁—S—S—R₂—Y
wherein R₁ and R₂ independently comprise a spacer comprising n carbon atoms, wherein n comprises an integer greater than 12, wherein X comprises: 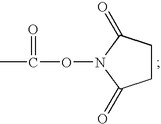
and wherein Y comprises an organic group; and wherein a recognition molecule capable of recognizing the analyte is covalently bonded to X; and
measuring an electrical signal via the transducer, wherein the electrical signal correlates with a concentration of the analyte in the sample.
